(19) United States
(12) Patent Application Publication
(10) Pub. No.: US 2015/0340129 A1
(43) Pub. Date: Nov. 26, 2015

(54) HARNESS

(71) Applicant: Hitachi Metals, Ltd., Tokyo (JP)

(72) Inventors: Detian HUANG, Hitachi (JP); Takanobu Watanabe, Hitachi (JP); Kimika Kudo, Kitaibaraki (JP)

(21) Appl. No.: 14/505,618

(22) Filed: Oct. 3, 2014

(30) Foreign Application Priority Data
May 22, 2014 (JP) 2014-105998

(51) Int. Cl. H01B 7/42; H01B 7/00; A61B 8/00

(52) U.S. Cl. CPC H01B 7/421; A61B 8/56; H01B 7/0045; A61B 2562/222

(57) ABSTRACT

A harness includes a cable having a braid shield, and a terminal component including a heat generating part disposed at least one end of the cable. The braid shield includes braided rectangular element wires each having a rectangular cross section. The terminal component includes a caulking ring to caulk and fix the braid shield and to be fixed to a housing of the terminal component, and a heat collection metal pipe thermally connected to the caulking ring and extended to a vicinity of the heat generating part.

The present application is based on Japanese patent application No. 2014-105998 filed on May 22, 2014, the entire contents of which are incorporated herein by reference.

### BACKGROUND OF THE INVENTION

1. Field of the Invention

This invention relates to a harness. In particular, this invention relates to a harness that is capable of dissipating heat generated in a terminal component disposed in a cable terminal.

2. Description of the Related Art

A probe cable for an ultrasonic diagnostic device is known in which plural core wires (i.e., coaxial wires and insulated wires) are bundled with a bind tape and a braid shield and a sheath layer are sequentially disposed on the outer periphery of the bundled core wires.

For ultrasonic diagnostic devices, a problem is how to dissipate heat generated in a probe according to an increase in medical examination frequency (i.e., increase in use time).

A method is conventionally known that an element wire comprised of a soft copper wire is used for a braid shield, and the element wires are connected to a ground pattern of a substrate in the probe so as to thereby dissipate outside heat generated in the probe via the braid shield.

Another method is known that a cooling tube is inserted into the cable and a cooling liquid is circulated through the cooling tube so as to thereby dissipate heat (e.g., refer to JP-A-2007-188737).

### SUMMARY OF THE INVENTION

The probe cable for an ultrasonic diagnostic device is repeatedly bent, thus it is required to have a long bending disconnection life, and for the purpose of enhancement in operability, it is also required to be light in weight and easily bent.

However, in the conventional method to dissipate heat via the braid shield, a problem may arise that not only sufficient heat dissipation effect cannot be obtained, but also when repeatedly bent, the element wires of the braid shield are easily disconnected, thus the bending disconnection life shortens.

Also, in the method to use the cooling tube, a problem may arise that due to the fact that the cooling tube is inserted, the cable becomes thick, hard and heavy in weight, so that the operability thereof is deteriorated. Another problem may arise that the cooling effect may not be obtained for the reason that the cooling liquid is lost by aging etc. Further, the method needs a device etc. to circulate the cooling liquid, thus the cost increases.

It is an object of the invention to provide a harness that is adapted to efficiently dissipate heat generated in a terminal component and allows a cable included therein to have a long bending disconnection life and to be lightweight and easily bent.

(1) According to one embodiment of the invention, a harness comprises:

a cable having a braid shield; and

a terminal component comprising a heat generating part disposed at least one end of the cable,

wherein the braid shield comprises braided rectangular element wires each having a rectangular cross section, and

wherein the terminal component comprises a caulking ring to caulk and fix the braid shield and to be fixed to a housing of the terminal component, and a heat collection metal pipe thermally connected to the caulking ring and extended to a vicinity of the heat generating part.

In the above embodiment (1) of the invention, the following modifications and changes can be made.

(i) The braid shield is braided such that the braiding rectangular element wires are brought into surface contact with each other.

(ii) The rectangular element wires are formed by rolling element wires each having a circular cross section.

(iii) The cable is configured such that the braid shield is disposed so as to collectively cover a plurality of coaxial wires.

### EFFECTS OF THE INVENTION

According to one embodiment of the invention, a harness can be provided that is adapted to efficiently dissipate heat generated in a terminal component and allows a cable included therein to have a long bending disconnection life and to be lightweight and easily bent.

### BRIEF DESCRIPTION OF THE DRAWINGS

The preferred embodiments according to the invention will be explained below referring to the drawings, wherein:

FIG. 1A is a perspective view schematically showing a harness according to one embodiment of the invention;

FIG. 1B is a cross-sectional view schematically showing a cable used for the embodiment of the invention; and

FIG. 1C is an explanatory view schematically showing a braid shield used for the embodiment of the invention.

### DETAILED DESCRIPTION OF THE PREFERRED EMBODIMENTS

Hereinafter, the embodiments according to the invention will be explained below referring to the drawings.

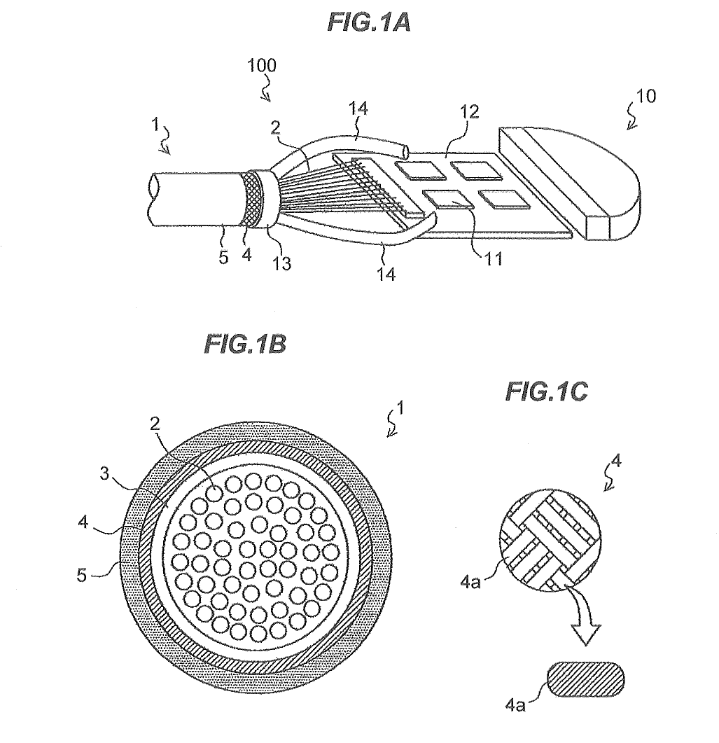

FIG. 1A is a perspective view schematically showing a harness according to one embodiment of the invention, FIG. 1B is a cross-sectional view schematically showing a cable used for the embodiment of the invention and FIG. 1C is an explanatory view schematically showing a braid shield used for the embodiment of the invention. As shown in FIGS. 1A to 1C, a harness 100 is configured to include a cable 1 having a braid shield 4 and a probe head as a terminal component 10 having heat generating parts 11 disposed in at least one end part of the cable 1.

The cable 1 is configured such that a plurality of core wires (coaxial wires and insulated wires) 2 are bundled with a bind tape 3, and a braid shield 4 and a sheath layer 5 are sequentially disposed on the outer periphery of the bundled core wires. The cable 1 is, for example, a probe cable used in an ultrasonic diagnostic device for connecting a device side and a probe side, and the total number of the core wires 2 is, for example, not less than 100.

The bind tape 3 is a resin tape used for bundling a plurality of the core wires 2, and as the bind tape 3, a polytetrafluoroethylene (PTFE) tape can be used.

The sheath layer 5 is comprised of an insulating resin for medical use. The insulating resin for medical use means a resin having a high biocompatibility such as a resin having no toxicity and preventing occurrence of the allergic symptoms such as inflammation when brought into contact with human bodies, and a resin being also called a medical resin or a resin of medical grade. In the embodiment, as the insulating resin for medical use used for the sheath layer 5, polyvinylchloride (PVC) of medical grad is used.

In the harness 100 according to the embodiment, the braid shield 4 is formed by braiding rectangular element wires 4a having an approximately rectangular cross section.

In order to prevent damages of the bind tape 3 and the sheath layer 5, it is preferable that the rectangular element wires 4a of which corners are rounded and which are formed by rolling element wires having a circular cross section are used for the braid shield 4.

The braid shield 4 is formed by braiding the braiding rectangular element wires 4a so that the braiding rectangular element wires 4a are brought into surface contact with each other. In the embodiment, the rectangular element wires 4a having an approximately rectangular shape in a cross sectional view are used, and the braid shield 4 is formed by braiding the rectangular element wires 4a so that the surfaces constituting the long sides of the rectangular element wires 4a in cross sectional view are brought into surface contact with each other. According to the above-mentioned configuration, it becomes possible to markedly enlarge the contact area between the element wires in comparison with a configuration that element wires having a circular cross section are used, so that an efficiency of heat conduction in the braid shield 4 can be drastically enhanced and a heat dissipation effect can be enhanced.

It is preferable that a soft copper wire having a high heat conductivity is used as the rectangular element wires 4a. In addition, at the time of braiding the rectangular element wires 4a, it is preferable that the braiding is carried out so as to form a braid having an angle of 30 to 45 degrees and a density of 80 to 90%. If the angle at the time of braiding is less than 30 degrees, when the cable 1 is bent, the rectangular element wires 4a are easily disconnected, and if the angle is more than 45 degrees, it becomes difficult to bend the cable 1. In addition, if the density at the time of braiding is less than 80%, air space is enlarged so that sufficient shield effect cannot be obtained and a heat dissipation effect is reduced, and if the density is more than 90%, the density becomes excessively high, thus it becomes difficult to bend the cable 1.

It is preferable that the rectangular element wire 4a is configured to have a thickness of 0.1 to 0.15 mm. If the thickness of the rectangular element wires 4a is less than 0.1 mm, the rectangular element wires 4a are easily broken, and if the thickness is more than 0.15 mm, the cable 1 becomes hard as a whole and the external form thereof is enlarged.

In addition, it is preferable that the rectangular element wire 4a is configured to have a width of approximately 0.5 to 2 mm according to the setting of the braided number at the time of braiding. If the width of the rectangular element wires 4a is more than 2 mm, when the cable 1 is bent, the rectangular element wires 4a are easily broken.

The core wires 2 of the cable 1 are connected to the internal substrate 12 of the terminal component 10. The internal substrate 12 is comprised of a printed circuit board (PCB) or a flexible printed circuit (FPC), and the heat generating parts 11 such as IC are mounted on the internal substrate 12.

In the harness 100 according to the embodiment, the terminal component 10 includes the caulking ring 13 configured to caulk and fix the braid shield 4 and simultaneously configured to be fixed to a housing (not shown) of the terminal component 10, and the metal pipe 14 for collection of heat configured to be thermally connected to the caulking ring 13 and configured to extend to the vicinity of the heat generating parts 11.

According to the above-mentioned configuration, heat generated in the heat generating parts 11 is transmitted to the braid shield 4 via the metal pipe 14 and the caulking ring 13, and is dissipated via the braid shield 4 to the outside of the terminal component 10.

As the metal pipe 14, a pipe comprised of a metal having a high heat conductivity such as copper, aluminum is preferably used. The metal pipe 14 has a large surface area, thus it has a large heat collection effect, and the metal pipe 14 makes it possible to efficiently collect the heat generated in the heat generating parts 11 and to transmit the heat to the braid shield 4. The metal pipe 14 can be directly brought into contact with the heat generating parts 11, or does not necessarily have to be brought into contact with the heat generating parts 11. In addition, a configuration can be also adopted that the metal pipe 14 and the heat generating parts 11 are brought into contact with each other via a radiation sheet or the like.

Here, a configuration that two metal pipes 14 are used is shown, but the number of the metal pipe 14 is not particularly limited to the above-mentioned number.

In addition, here, the cable 1 and the metal pipes 14 are caulked and fixed by the caulking ring 13, thereby the caulking ring 13 and the metal pipes 14 are thermally connected to each other, but not particularly limited to this, for example, the caulking ring 13 and the metal pipes 14 may be integrally formed, or the caulking ring 13 and the metal pipes 14 may be thermally connected to each other by indirectly bringing the caulking ring 13 and the metal pipes 14 into contact with each other via the other component, for example, a housing.

As explained above, in the harness 100 according to the embodiment, the braid shield 4 is formed by braiding rectangular element wires 4a having a rectangular cross section, and the terminal component 10 includes the caulking ring 13 configured to caulk and fix the braid shield 4 and simultaneously configured to be fixed to a housing of the terminal component 10, and the metal pipe 14 for collection of heat configured to be thermally connected to the caulking ring 13 and configured to extend to the vicinity of the heat generating parts 11.

The above-mentioned configuration makes it possible that heat generated in the terminal component 10 is efficiently collected by the metal pipe 14 and the collected heat is transmitted to the braid shield 4 via the caulking ring 13, and furthermore the heat is efficiently transmitted by the braid shield 4 using the rectangular element wires 4a so as to be efficiently dissipated via the braid shield 4 to the outside of the terminal component 10.

In a configuration that element wires having a circular cross section are used for the braid shield 4, the element wires are brought into point contact with each other, thus the contact area between the element wires is small, but in a configuration that the rectangular element wires 4a are used for the braid shield 4, the element wires are brought into surface contact with each other, thus the contact area between the element wires in the whole of the braid shield 4 is markedly increased, so that heat dissipation effect can be enhanced.

In addition, in the embodiment, the rectangular element wires 4a are used for the braid shield 4, thus when the cable 1 is bent, disconnection does not easily occur in comparison with a configuration that element wires having a circular cross section are used, so that bending disconnection life can be enhanced. Furthermore, the rectangular element wires 4a are used for the braid shield 4, thereby it becomes possible to reduce the thickness of the braid shield 4 so as to reduce the outer diameter of the whole of the cable 1, so that a high flexibility can be realized.

Furthermore, in the embodiment, heat dissipation effect can be enhanced without adding the other component such as a cooling tube thereto, thus the harness 100 configured to be light in weight, easily bent and high in operability can be realized in comparison with a configuration that the other component such as a cooling tube is added thereto. In addition, in the embodiment, a device for circulating a cooling liquid or the like is not needed, thus cost is low.

As mentioned above, according to the embodiment, the heat generated in the terminal component 10 can be efficiently dissipated, and the harness 100 configured such that the cable 1 disposed therein has a long bending disconnection life, and is light in weight and easily bent can be realized.

Although the invention has been described with respect to the specific embodiments for complete and clear disclosure, the appended claims are not to be thus limited but are to be construed as embodying all modifications and alternative constructions that may occur to one skilled in the art which fairly fall within the basic teaching herein set forth.

For example, in the above-mentioned embodiment, a configuration that the terminal component 10 is a probe head has been explained, but the terminal component 10 is not particularly limited to this, for example, the terminal component 10 can be a substrate or a connector.

In addition, in the above-mentioned embodiment, a configuration that the cable 1 is a probe cable having a plurality of core wires 2 has been explained, but the cable 1 is not particularly limited to this, and if the cable 1 is configured to include the braid shield 4, any cable 1 can be applied.

What is claimed is:

1. A harness, comprising:
a cable having a braid shield; and
a terminal component comprising a heat generating part disposed at least one end of the cable,
wherein the braid shield comprises braided rectangular element wires each having a rectangular cross section, and
wherein the terminal component comprises a caulking ring to caulk and fix the braid shield and to be fixed to a housing of the terminal component, and a heat collection metal pipe thermally connected to the caulking ring and extended to a vicinity of the heat generating part.

2. The harness according to claim 1, wherein the braid shield is braided such that the braiding rectangular element wires are brought into surface contact with each other.

3. The harness according to claim 1, wherein the rectangular element wires are formed by rolling element wires each having a circular cross section.

4. The harness according to claim 1, wherein the cable is configured such that the braid shield is disposed so as to collectively cover a plurality of coaxial wires.
